(19) United States
(12) Patent Application Publication
(10) Pub. No.: US 2014/0180042 A1
(43) Pub. Date: Jun. 26, 2014

(54) Methods and Systems for Detecting a Sensor Off Condition Using A Reference Ambient Characteristic

(71) Applicant: COVIDIEN LP, Mansfield, MA (US)

(72) Inventors: Paul Stanley Addison, Edinburgh (GB); James Nicholas Watson, Dunfermline (GB)

(73) Assignee: Covidien LP, Mansfield, MA (US)

(21) Appl. No.: 13/723,150

(22) Filed: Dec. 20, 2012

(51) Int. Cl. A61B 5/0205; A61B 5/00; A61B 5/1455

(52) U.S. Cl. CPC A61B 5/0205; A61B 5/14551; A61B 5/7221; A61B 5/6831; A61B 5/6832; A61B 5/6815; A61B 5/6824; A61B 5/6826; A61B 5/6829
USPC 600/324

(57) ABSTRACT

A physiological monitoring system may use photonic signals at one or more wavelengths to determine physiological parameters. During monitoring, a physiological sensor may become improperly positioned, which may affect the physiological attenuation of the photonic signals, and accordingly a detected light signal. The detected light signal may include an ambient light component and a signal component corresponding to the one or more wavelengths of light. The physiological monitoring system may determine a reference characteristic based on the ambient light component, and compare the signal component with the ambient light component to determine a sensor-off condition.

The present disclosure relates to detecting a sensor condition, and more particularly relates to detecting a sensor off condition in a pulse oximeter or other medical device using a reference ambient characteristic.

### SUMMARY

Methods and systems are provided for determining whether a physiological sensor is properly positioned on a subject.

In some embodiments, a method may be provided for determining whether a physiological sensor is properly positioned on a subject. The method may include receiving a detected light signal including an ambient light signal component and a signal component corresponding to a wavelength of light emitted by the physiological sensor. The method may also include processing the detected light signal to generate a first signal corresponding to the ambient light signal component, and processing the detected light signal to generate a second signal corresponding to the ambient light signal component and the signal component. The method may further include determining at least one reference characteristic based on the first signal, comparing the second signal to the at least one reference characteristic, and determining whether the physiological sensor is properly positioned based on the comparison.

In some embodiments, a system may be configured for determining whether a physiological sensor is properly positioned on a subject. The system may include processing equipment configured to receive a detected light signal including an ambient light signal component and a signal component corresponding to a wavelength of light emitted by the physiological sensor. The processing equipment may be configured to process the detected light signal to generate a first signal corresponding to the ambient light signal component, and process the detected light signal to generate a second signal corresponding to the ambient light signal component and the signal component. The processing equipment may be further configured to determine at least one reference characteristic based on the first signal, compare the second signal to the at least one reference characteristic, and determine whether the physiological sensor is properly positioned based on the comparison.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system, in accordance with some embodiments of the present disclosure;

FIG. 2A shows an illustrative plot of a light drive signal, in accordance with some embodiments of the present disclosure;

FIG. 2B shows an illustrative plot of a detector signal, in accordance with some embodiments of the present disclosure;

FIG. 3 is a perspective view of an embodiment of a physiological monitoring system, in accordance with some embodiments of the present disclosure;

FIG. 4 shows an illustrative signal processing system, in accordance with some embodiments that may implement the signal processing techniques described herein;

FIG. 5 is a flow diagram showing illustrative steps for detecting a sensor-off condition, in accordance with some embodiments of the present disclosure;

FIG. 6 shows an illustrative plot of an IR signal, an ambient baseline and a threshold, in accordance with some embodiments of the present disclosure;

FIG. 7 shows an illustrative plot of an fluctuating IR signal, an ambient baseline and thresholds, in accordance with some embodiments of the present disclosure; and

FIG. 8 shows illustrative plots of an IR signal, an ambient signal component, and a threshold, along with a sensor-off flag, in accordance with some embodiments of the present disclosure.

### DETAILED DESCRIPTION OF THE FIGURES

The present disclosure is directed towards detecting a sensor off condition in a medical device. A physiological monitoring system may monitor one or more physiological parameters of a patient, typically using one or more physiological sensors. For example, the physiological monitoring system may include a pulse oximeter. In a further example the physiological monitoring system may be configured to determine blood oxygen saturation, pulse rate, respiration rate, respiration effort, continuous non-invasive blood pressure (CNIBP), saturation pattern detection, fluid responsiveness, cardiac output, or any other suitable physiological parameter that may be determined using a pulse oximeter. The system may include, for example, a light source and a photosensitive detector. In some embodiments, a sensor may be attached to a target area of a patient. For example, the sensor may be attached using an adhesive, a strap, a band, elastic, any other suitable attachment, or any combination thereof. In some embodiments, the sensor may be located proximate to a desired structural element. For example, a sensor may be held near to the radial artery using a wrist strap. In another example, a sensor may be held near to the blood vessels of the forehead using an adhesive or tape. The techniques disclosed herein may be applied to any suitable sensor such as, for example, finger sensors, ear sensors, toe sensors, forehead sensors, or any other suitable sensor that senses an ambient or “dark” signal.

In some embodiments, the system may detect a sensor-off condition. As used herein, the sensor-off condition may include any condition where the sensor is fully or partially detached or moved from the desired target area of the subject. A sensor-off condition may include a condition where an adhesive coupling the sensor to the subject has fully or partially failed. A sensor-off condition may include a condition where a sensor held with a strap or band has loosened, shifted, slid, moved, detached, repositioned in any other unsuitable arrangement, or any combination thereof. For example, a sensor held by an adhesive to the forehead of a subject may fully or partially separate due to an adhesive failure, resulting in a sensor-off condition. In another example, a sensor held proximal to the radial artery at the wrist of a subject by a strap or band may shift out of position, resulting in a sensor-off position. It will be understood that the sensor-off conditions described here are merely exemplary and that any suitable undesirable positioning of the sensor may result in a sensor-off condition. It will also be understood that the particular arrangement of a sensor-off condition may dependent upon the configuration and type of sensor.

The sensor-off condition may be detected by the system. In some embodiments, the system may use an ambient light signal to determine a sensor-off condition. As will be described in detail below, an ambient light signal may include the amount of light a detector receives when one or more associated light sources are in an “off” state. In some embodiments where a detector receives light from a light sources coupled to the system and from light sources not coupled to the system, the ambient light signal may include light from light sources not coupled to the system. Ambient light sources may include sunlight, incandescent room lights, fluorescent room lights, fireplaces, candles, naked flames, LED room lights, instrument panel lighting, any other suitable light sources not intended for determining a physiological parameter, or any combination thereof. In some embodiments, the ambient light signal may include decaying LED light from the system light sources. For example, it may take a particular amount of time for the light output from a light source to decrease to zero following the light drive signal being switched off. A portion of this emitted light may be included in the ambient signal. In some embodiments, the ambient light signal may not contain physiological information.

In some embodiments, a sensor may be designed to limit the amount of ambient light received by a detector. For example, a detector may be arranged close to and facing the skin. A detector may include a light blocking material between the detector and an ambient light source, to prevent ambient light from reaching the detector. In a further example, a system may include other suitable shields, optics, filters, arrangements, or any combination thereof, to reduce ambient light signals received by the receiver. In some embodiments, the particular arrangement of light blocking structures or material may depend on the type of sensor. For example, a forehead sensor may include flat light blocking structure, while a fingertip sensor may include a light blocking structure that encircles the finger.

It will be understood, however, that many clinical settings include relatively bright light sources and the ambient light signals received by the detector may not necessarily be zero when the sensor is positioned as desired. Similarly, shielding ambient light may be more difficult for a forehead sensor than, for example, a fingertip sensor.

In some embodiments, for example, a fingertip sensor where light may be generated by the system on one side of a finger and detected on the opposite side of a finger, removing the detector from a finger (i.e., a sensor-off condition) may result in all of the generated light being received by the sensor, rather than a portion of the light being attenuated by interacting with the tissue of the subject. This relatively high signal level may be detected as a sensor-off condition by the system.

In some circumstances, for example, a sensor-off condition need not necessarily result in a relatively high detected signal level. A forehead sensor may include a light source placed relatively close to a detector on the forehead of a patient using tape, an adhesive, a band encircling the skull, any other suitable arrangement, or any combination thereof. The light source and detector may be arranged such that a portion of the light emitted from the light source interacts with, and is partially attenuated by, the tissue of the subject and the attenuated light is detected by the detector. The light source may be pulsed, such that an ambient light signal is detected by the detector between the pulses, and a total signal detected during the pulses includes both the ambient and the desired light. In determining a physiological parameter, the ambient light signal may be, for example, subtracted from the total signal. In some embodiments, the ambient signal may exhibit characteristic behavior of a sensor-off condition. In some embodiments, the ambient light signal may remain relatively constant with respect to certain system changes. For example, the ambient light signal may be relatively insensitive to changes in physiological conditions.

In some embodiments, the proximity of a signal component corresponding to a wavelength of light emitted by the sensor (e.g., an IR signal component, a Red signal component) relative to a characteristic or baseline ambient light (AM) signal, sometimes also referred to as a “dark signal”, may be monitored to determine whether a sensor is positioned properly. The AM signal and its relationship to the signal component may exhibit characteristic behavior distinctive of the sensor's position. The techniques disclosed herein may identify a Sensor Off condition for certain conditions where the signal component corresponding to a wavelength of light emitted by the sensor does not substantially mimic the behavior of the AM signal, but rather fluctuates or otherwise trends relative to the AM signal.

In some embodiments, a baseline AM signal value may be derived. The baseline AM signal value may be derived, for example, when the sensor is first attached to the subject (e.g., an initial value may be used as the baseline). For example, forehead sensors typically exhibit good shielding values, which aids in acquiring a useful measurement of an AM baseline value. In some embodiments, the signal component corresponding to a wavelength of light emitted by a sensor may be tracked in time, and a Sensor Off condition may be flagged if the signal component is reduced relative to a baseline AM value. In some embodiments, a threshold may be generated based on the AM signal, and a Sensor Off condition may be flagged if the signal component crosses the threshold.

In some embodiments, the relative proximity of the signal component corresponding to a wavelength of light emitted by a sensor to the AM baseline signal may indicate a fluctuating signal indicative of a Sensor Off condition. In some circumstances, a non-constant AM and/or signal component may be exhibited. For example, non-constant character may be exhibited when the sensor moves relative to a reflective surface, the sensor is moving relative to a shading material, or the LED and photodetectors are moving relative to each other. In some embodiments, the fluctuations may be quantified and used to determine whether a sensor is positioned properly.

The disclosed techniques are particularly valuable, for example, where there is weak or no ambient light and an IR signal or Red signal during detachment is relatively small. This may occur for a forehead sensor, for example, when the LED light does not shine directly or indirectly (e.g., by reflection) onto the photodiode during detachment. This may also occur for other sensors (e.g., disposable finger sensors), where the LED light may not shine onto the photodiode during a sensor off state.

An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is selected to be of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to determine ambient signal levels, reduce overlap of the light drive pulses, allow time for light sources to stabilize, reduce heating effects, reduce power consumption, for any other suitable reason, or any combination thereof.

It will be understood that the sensor-off techniques described herein are not limited to pulse oximeters and may be applied to any suitable medical and non-medical devices. For example, the system may include sensors for regional saturation (rSO2), respiration rate, respiration effort, continuation non-invasive blood pressure, saturation pattern detection, fluid responsiveness, cardiac output, any other suitable clinical parameter, or any combination thereof. Sensors may be used with a pulse oximeter, a general purpose medical monitor, any other suitable medical device, or any combination thereof. In some embodiments, the sensor-off identification techniques described herein may be applied to analysis of light levels where an ambient or dark signal may be required.

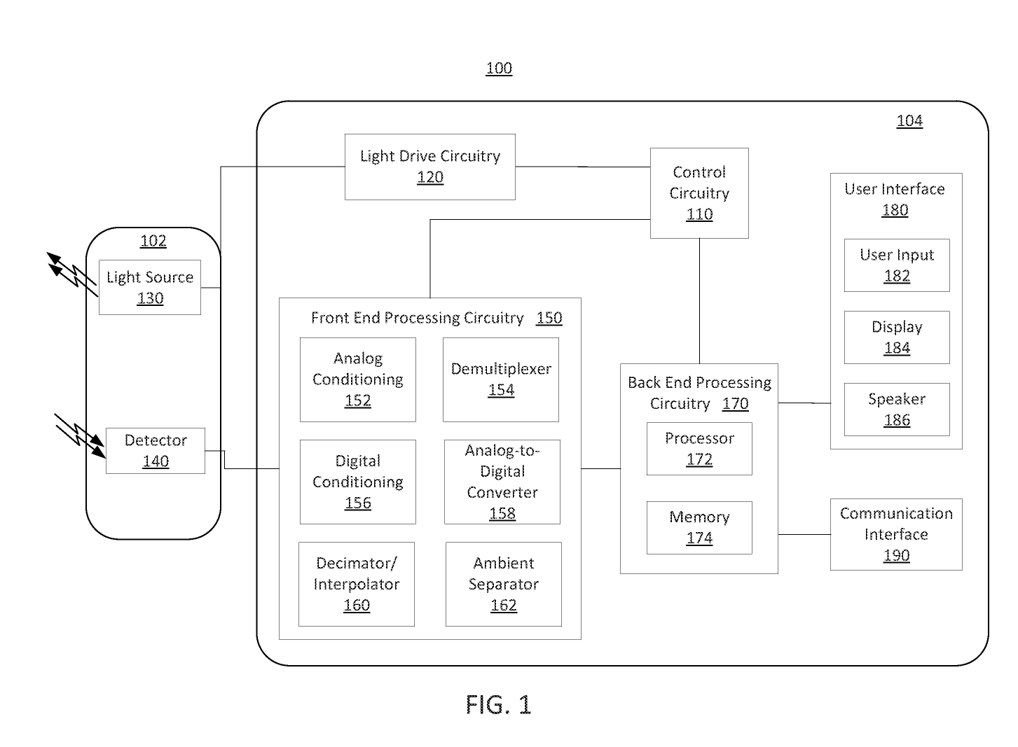

FIG. 1 is a block diagram of an illustrative physiological monitoring system 100 in accordance with some embodiments of the present disclosure. System 100 may include a sensor 102 and a monitor 104 for generating and processing physiological signals of a subject. In some embodiments, sensor 102 and monitor 104 may be part of an oximeter.

Sensor 102 of physiological monitoring system 100 may include light source 130 and detector 140. Light source 130 may be configured to emit photonic signals having one or more wavelengths of light (e.g. Red and IR) into a subject's tissue. For example, light source 130 may include a Red light emitting light source and an IR light emitting light source, e.g., Red and IR light emitting diodes (LEDs), for emitting light into the tissue of a subject to generate physiological signals. In one embodiment, the Red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a Red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the Red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed, scattered, or reflected, less light of that wavelength is typically received from the tissue by detector 140. After converting the received light to an electrical signal, detector 140 may send the detection signal to monitor 104, where the detection signal may be processed and physiological parameters may be determined (e.g., based on the absorption of the Red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals of control circuitry 110 to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals of control circuitry 110 to coordinate its operation with front end processing circuitry 150.

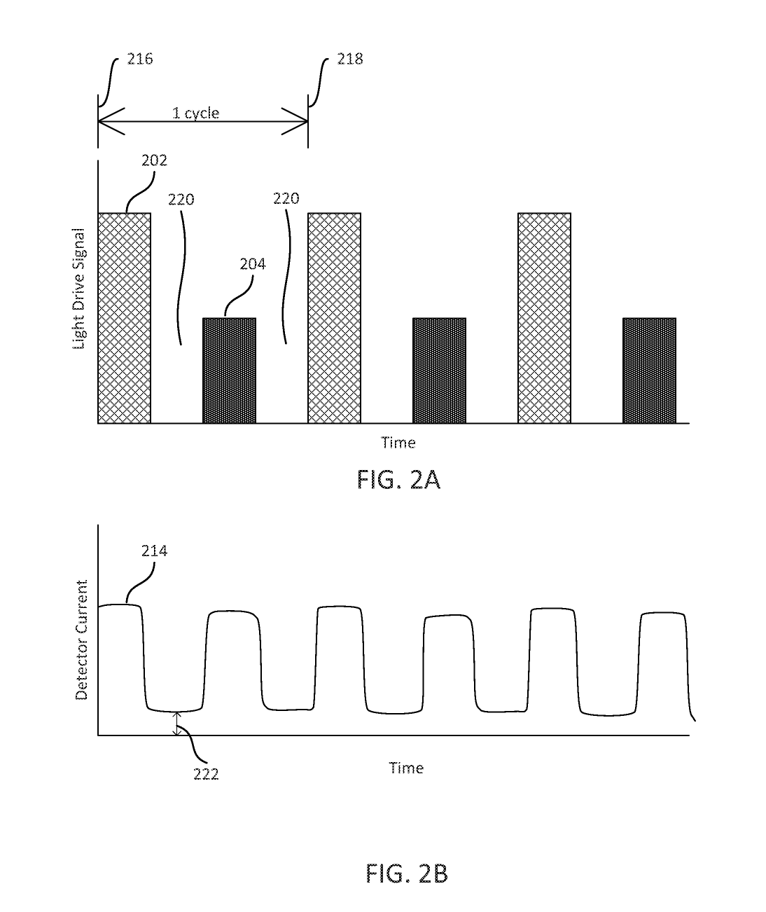

Light drive circuitry 110, as discussed above, may be configured to generate a light drive signal that is provided to light source 130 of sensor 102. The light drive signal may, for example, control the intensity of light source 130 and the timing of switching light source 130 on and off. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

FIG. 2A shows an illustrative plot of a light drive signal including red light drive pulse 202 and IR light drive pulse 204 in accordance with some embodiments of the present disclosure. Drive pulses 202, and 204 may be generated by light drive circuitry 120 under the control of control circuitry 110. As used herein, drive pulses may refer to switching power or other components on and off, high and low output states, high and low values within a continuous modulation, other suitable relatively distinct states, or any combination thereof. The light drive signal may be provided to light source 130, including red drive pulse 202 and IR drive pulse 204 to drive red and IR light emitters, respectively, within light source 130. Red drive pulse 202 may have higher amplitude than IR drive 204 since red LEDs may be less efficient than IR LEDs at converting electrical energy into light energy. In some embodiments, the output levels may be the equal, may be adjusted for nonlinearity of emitters, may be modulated in any other suitable technique, or any combination thereof. Additionally, red light may be absorbed and scattered more than IR light when passing through perfused tissue. When the red and IR light sources are driven in this manner they emit pulses of light at their respective wavelengths into the tissue of a subject in order generate physiological signals that physiological monitoring system 100 may process to calculate physiological parameters. It will be understood that the light drive amplitudes of FIG. 2A are merely exemplary and any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the Red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. Similarly, the signal from detector 140 may require time to decay completely to a final state after light source 130 is switched off. The period from time 216 to time 218 may be referred to as a drive cycle, which includes four segments: a Red light drive pulse 202, followed by an “off” period 220 in FIG. 2A, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each Red light drive pulse 202 and each IR drive pulse 204 may be understood to be surrounded by two “off” periods 220 in FIG. 2A. “Off” periods may also be referred to as dark periods, in that the emitters are dark during that period.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector signal 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current waveform 214 may be generated in response to a light source being driven by the light drive signal of FIG. 2A. The valleys of detector current waveform 214 may be synchronous with periods of time during which no light is being emitted by the light source. While no light is being emitted by a light source during the valleys, detector current waveform 214 need not decrease to zero. Rather, ambient signal 222 may be present in the detector waveform, as well as other background amplitude contributions. In some embodiments, ambient signal 222 may be used to determine a sensor-off condition. In some embodiments, ambient signal 222 may be removed from a processed signal to facilitate determination of physiological parameters.

Referring back to FIG. 1, front end processing circuitry 150, which may receive a detection signal, such as detector current waveform 214, may include analog conditioner 152, demultiplexer 154, digital conditioner 156, analog-to-digital converter (ADC) 158, decimator/interpolator 160, and ambient subtractor 162.

In some embodiments, front end processing circuitry 150 may include a second analog-to-digital converter (not shown) configured to sample the unprocessed detector signal. This signal may be used to detect changes in the ambient light level without applying the signal condition and other steps that may improve the quality of determined physiological parameters but may reduce the amount of information regarding a sensor-off condition.

Analog conditioner 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signal may be processed by analog-to-digital converter 158, which may convert the conditioned analog signal into a digital signal. Analog-to-digital converter 158 may operate under the control of control circuitry 110. Analog-to-digital converter 158 may use timing control signals from control circuitry 110 to determine when to sample the analog signal. Analog-to-digital converter 158 may be any suitable type of analog-to-digital converter of sufficient resolution to enable a physiological monitor to accurately determine physiological parameters.

Demultiplexer 154 may operate on the analog or digital form of the detector signal to separate out different components of the signal. For example, detector current waveform 214 of FIG. 2B includes a Red component, an IR component, and at least one ambient component. Demultiplexer 154 may operate on detector current waveform 214 of FIG. 2B to generate a Red signal, an IR signal, a first ambient signal (e.g., corresponding to the ambient component that occurs immediately after the Red component), and a second ambient signal (e.g., corresponding to the ambient component that occurs immediately after the IR component). Demultiplexer 154 may operate under the control of control circuitry 110. For example, demultiplexer 154 may use timing control signals from control circuitry 110 to identify and separate out the different components of the detector signal.

Digital conditioner 156 may perform any suitable digital conditioning of the detector signal. Digital conditioner 156 may perform any type of digital filtering of the signal (e.g., low pass, high pass, band pass, notch, or any other suitable filtering), amplifying, perform an operation on the signal, perform any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove ambient values from the Red and IR components. In some embodiments, the system may subtract the ambient values from the Red and IR components to generate adjusted Red and IR signals. For example, ambient subtractor 162 may determine a subtraction amount from the ambient signal portion of the detection signal and subtract it from the peak portion of the detection signal in order to reduce the effect of the ambient signal on the peak. For example, in reference to FIG. 2A, a detection signal peak corresponding to red drive pulse 202 may be adjusted by determining the amount of ambient signal during the “off” period 220 preceding red drive pulse 202. The ambient signal amount determined in this manner may be subtracted from the detector peak corresponding to red drive pulse 202. Alternatively, the “off” period 220 after red drive pulse 202 may be used to correct red drive pulse 202 rather than the “off” period 220 preceding it. Additionally, an average of the “off” periods 220 before and after red “on” period 202 may be used. In some embodiments, ambient subtractor 162 may output an ambient signal for further processing. Ambient subtractor 162 may average the ambient signal from multiple “off” periods 220, may apply filters or other processing to the ambient signal such as averaging filters, integration filters, delay filters, buffers, counters, any other suitable filters or processing, or any combination thereof.

It will be understood that in some embodiments, ambient subtractor 162 may be omitted. It will also be understood that in some embodiments, the system may not subtract the ambient contribution of the signal. It will also be understood that the functions of demultiplexer 154 and ambient subtractor 162 may be complementary, overlapping, combined into a single function, combined or separated in any suitable arrangement, or any combination thereof. For example, the received light signal may include an ambient signal, an IR light signal, and a red light signal. The system may use any suitable arrangement of demultiplexer 154 and ambient subtractor 162 to determine or generate any combination of: a red signal, an IR signal, a red ambient signal, an IR ambient signal, an average ambient signal, a red with ambient signal, an IR with ambient signal, any other suitable signal, or any combination thereof.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 158. This may be achieved by applying a gain to the detected signal using analog conditioner 152 to map the expected range of the detection signal to the full or close to full dynamic range of analog-to-digital converter 158. In some embodiments, the input to analog-to-digital converter 158 may be the sum of the detected light multiplied by an analog gain value.

Ideally, when ambient light is zero and when the light source is off, the analog-to-digital converter 158 will read just above the minimum input value. When the light source is on, the total analog gain may be set such that the output of analog-to-digital converter 158 may read close to the full scale of analog-to-digital converter 158 without saturating. This may allow the full dynamic range of analog-to-digital converter 158 to be used for representing the detection signal, thereby increasing the resolution of the converted signal. In some embodiments, the total analog gain may be reduced by a small amount so that small changes in the light level incident on the detector do not cause saturation of analog-to-digital converter 158.

Back end processing circuitry 170 may include processor 172 and memory 174. Processor 172 may be adapted to execute software, which may include an operating system and one or more applications, as part of performing the functions described herein. Processor 172 may receive and further process physiological signals received from front end processing circuitry 150. For example, processor 172 may determine one or more physiological parameters based on the received physiological signals. Memory 174 may include any suitable computer-readable media capable of storing information that can be interpreted by processor 172. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth. In an embodiment, the subject may be a medical patient and display 184 may exhibit a list of values which may generally apply to the patient, such as, for example, age ranges or medication families, which the user may select using user inputs 182. Additionally, display 184 may display, for example, an estimate of a subject's blood oxygen saturation generated by monitor 104 (referred to as an “SpO₂” measurement), pulse rate information, respiration rate information, blood pressure, sensor condition, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232 or other standards), wireless communication (e.g., using WiFi, IR, WiMax, BLUETOOTH, UWB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In some embodiments, the functionality of one or more of the components may be performed in a different order or may not be required. In some embodiments, all of the components of physiological monitoring system 100 can be realized in processor circuitry.

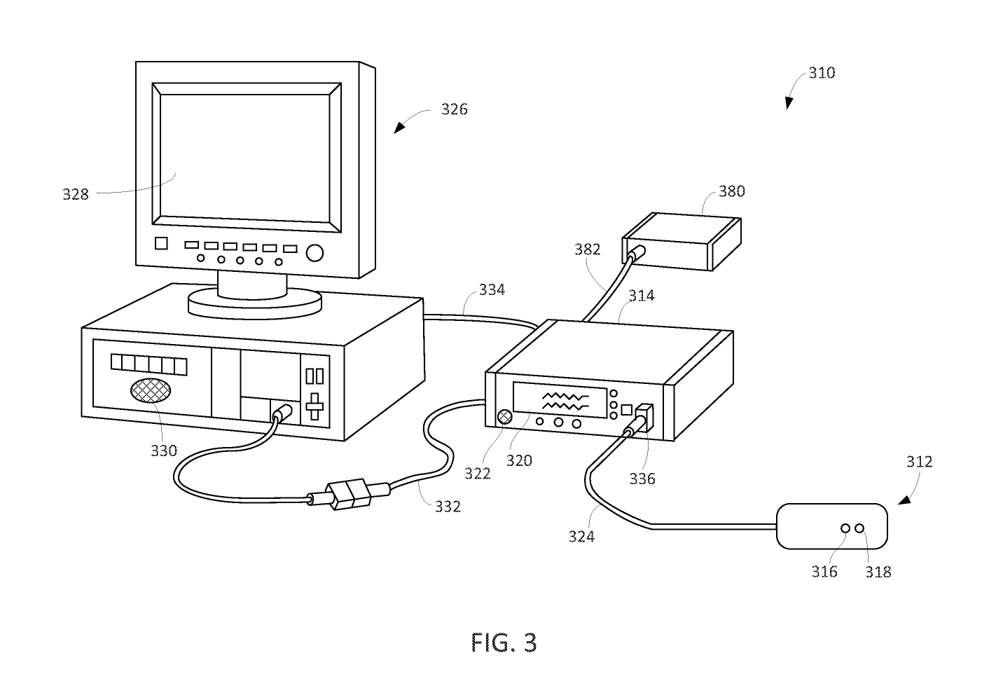

FIG. 3 is a perspective view of an embodiment of a physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two or more different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected to monitor 314 (not shown). Monitor 314 may be configured to calculate physiological parameters based at least in part on data relating to light emission and detection received from one or more sensor units such as sensor unit 312. For example, monitor 314 may be configured to determine pulse rate, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range or when a sensor is not properly positioned. In some embodiments, physiological monitoring system 310 includes a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological monitoring system).

In the illustrated embodiment, physiological monitoring system 310 includes a multi-parameter physiological monitor 326. The monitor 326 may include a display 328 including a cathode ray tube display, a flat panel display (as shown) such as a liquid crystal display (LCD) or a plasma display, any other suitable display, or any combination thereof. Multi-parameter physiological monitor 326 may be configured to calculate physiological parameters and to provide information from monitor 314 and/or from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, all or some of monitor 314 and multi-parameter physiological monitor 326 may be referred to collectively as processing equipment.

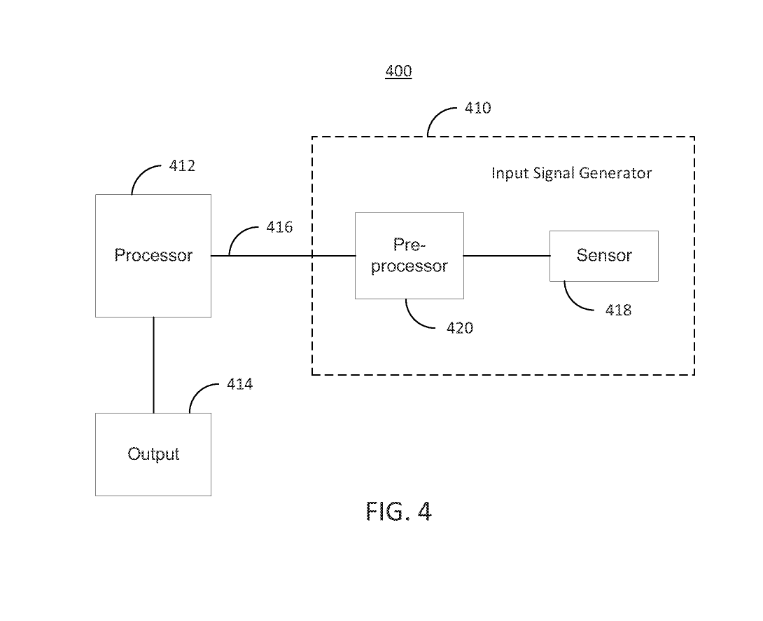

FIG. 4 shows illustrative signal processing system 400 in accordance with some embodiments of the present disclosure. Signal processing system 400 includes input signal generator 410, processor 412 and output 414. In the illustrated embodiment, input signal generator 410 may include pre-processor 420 coupled to sensor 418. As illustrated, input signal generator 410 generates an input signal 416. In some embodiments, input signal 416 may include one or more intensity signals based on a detector output. In some embodiments, pre-processor 420 may be an oximeter and input signal 416 may be a PPG signal. In an embodiment, pre-processor 420 may be any suitable signal processing device and input signal 416 may include PPG signals and one or more other physiological signals, such as an electrocardiogram (ECG) signal. It will be understood that input signal generator 410 may include any suitable signal source, signal generating data, signal generating equipment, or any combination thereof to produce signal 416. Signal 416 may be a single signal, or may be multiple signals transmitted over a single pathway or multiple pathways.

Pre-processor 420 may apply one or more signal processing operations to the signal generated by sensor 418. For example, pre-processor 420 may apply a pre-determined set of processing operations to the signal provided by sensor 418 to produce input signal 416 that can be appropriately interpreted by processor 412, such as performing A/D conversion. In some embodiments, A/D conversion may be performed by processor 412. Pre-processor 420 may also perform any of the following operations on the signal provided by sensor 418: reshaping the signal for transmission, multiplexing the signal, modulating the signal onto carrier signals, compressing the signal, encoding the signal, and filtering the signal. In some embodiments, pre-processor 420 may include a current-to-voltage converter (e.g., to convert a photocurrent into a voltage), an amplifier, a filter, and A/D converter, a demultiplexer, any other suitable pre-processing components, or any combination thereof. In some embodiments, pre-processor 420 may include one or more components from front end processing circuitry 150 of FIG. 1.

In some embodiments, signal 416 may include PPG signals corresponding to one or more light frequencies, such as an IR PPG signal, a Red PPG signal, and ambient light. In some embodiments, signal 416 may include signals measured at one or more sites on a subject's body, for example, a subject's finger, toe, ear, arm, or any other body site. In some embodiments, signal 416 may include multiple types of signals (e.g., one or more of an ECG signal, an EEG signal, an acoustic signal, an optical signal, a signal representing a blood pressure, and a signal representing a heart rate). Signal 416 may be any suitable biosignal or any other suitable signal.

In some embodiments, signal 416 may be coupled to processor 412. Processor 412 may be any suitable software, firmware, hardware, or combination thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may, for example, include an assembly of analog electronic components. Processor 412 may calculate physiological information. For example, processor 412 may compute one or more of a pulse rate, respiration rate, blood pressure, or any other suitable physiological parameter. Processor 412 may perform any suitable signal processing of signal 416 to filter signal 416, such as any suitable band-pass filtering, adaptive filtering, closed-loop filtering, any other suitable filtering, and/or any combination thereof. Processor 412 may also receive input signals from additional sources (not shown). For example, processor 412 may receive an input signal containing information about treatments provided to the subject. Additional input signals may be used by processor 412 in any of the calculations or operations it performs in accordance with processing system 400.

In some embodiments, all or some of pre-processor 420, processor 412, or both, may be referred to collectively as processing equipment.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store fiducial information or initialization information corresponding to physiological monitoring. In some embodiments, processor 412 may store physiological measurements or previously received data from signal 416 in a memory device for later retrieval. In some embodiments, processor 412 may store calculated values, such as a pulse rate, a blood pressure, a blood oxygen saturation, a fiducial point location or characteristic, an initialization parameter, or any other calculated values, in a memory device for later retrieval.

Processor 412 may be coupled to output 414. Output 414 may be any suitable output device such as one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into physiological monitoring system 100 of FIG. 1 in which, for example, input signal generator 410 may be implemented as part of sensor 102, or into physiological monitoring system 310 of FIG. 3 in which, for example, input signal generator 410 may be implemented as part of sensor unit 312 of FIG. 3, and processor 412 may be implemented as part of monitor 104 of FIG. 1 or as part of monitor 314 of FIG. 3. Furthermore, all or part of system 400 may be embedded in a small, compact object carried with or attached to the subject (e.g., a watch, other accessory, or a smart phone). In some embodiments, a wireless transceiver (not shown) may also be included in system 400 to enable wireless communication with other components of physiological monitoring systems 100 of FIGS. 1 and 310 of FIG. 3. As such, physiological monitoring systems 100 of FIGS. 1 and 310 of FIG. 3 may be part of a fully portable and continuous subject monitoring solution. In some embodiments, a wireless transceiver (not shown) may also be included in system 400 to enable wireless communication with other components of physiological monitoring systems 100 of FIGS. 1 and 310 of FIG. 3. For example, pre-processor 420 may output signal 416 over BLUETOOTH, 802.11, WiFi, WiMax, cable, satellite, Infrared, or any other suitable transmission scheme. In some embodiments, a wireless transmission scheme may be used between any communicating components of system 400. In some embodiments, system 400 may include one or more communicatively coupled modules configured to perform particular tasks. In some embodiments, system 400 may be included as a module communicatively coupled to one or more other modules.

It will be understood that the components of signal processing system 400 that are shown and described as separate components are shown and described as such for illustrative purposes only. In other embodiments the functionality of some of the components may be combined in a single component. For example, the functionality of processor 412 and pre-processor 420 may combined in a single processor system. Additionally, the functionality of some of the components shown and described herein may be divided over multiple components. Additionally, signal processing system 400 may perform the functionality of other components not show in FIG. 4. For example, some or all of the functionality of control circuitry 110 of FIG. 1 may be performed in signal processing system 400. In other embodiments, the functionality of one or more of the components may not be required. In an embodiment, all of the components can be realized in processor circuitry.

In some embodiments, any of the processing components and/or circuits, or portions thereof, of FIGS. 1, 3, and 4 may be referred to collectively as processing equipment. For example, processing equipment may be configured to amplify, filter, sample, and digitize input signal 416 (e.g., using an analog-to-digital converter), and calculate physiological information from the digitized signal. Processing equipment may be configured to generate light drive signals, amplify, filter, sample and digitize detector signals, and calculate physiological information from the digitized signal. In some embodiments, all or some of the components of the processing equipment may be referred to as a processing module.

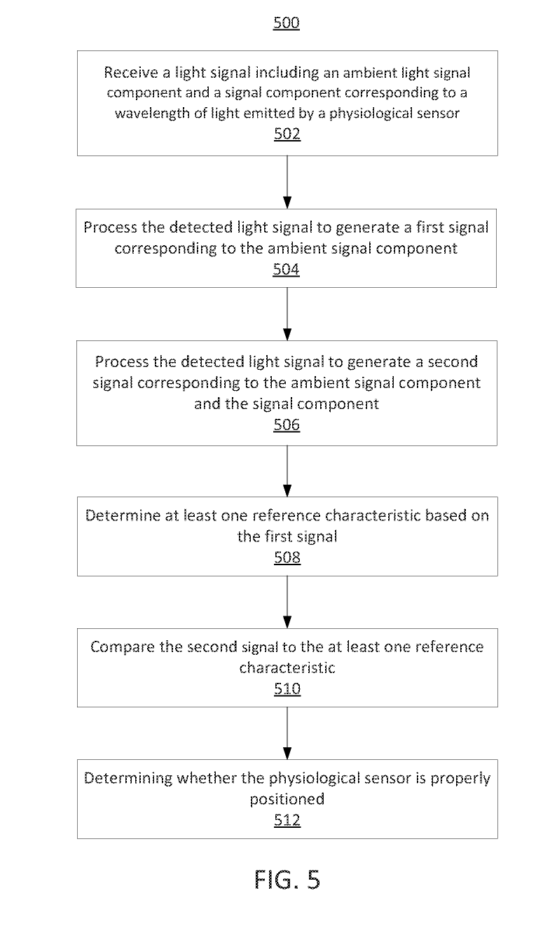

FIG. 5 is a flow diagram 500 of illustrative steps for detecting a sensor-off condition, in accordance with some embodiments of the present disclosure.

In step 502, the system may receive a detected light signal. The detected light signal may include light from drive pulses or other emitted light included in the emitted photonic signal that has interacted with the subject. The received light signal may be detected by, for example, detector 140 of FIG. 1. In some embodiments, a portion of the emitted light may be partially attenuated by the tissue of the subject before being received as a received light signal. In some embodiments, the received light may have been primarily reflected by the subject. For example, reflected light may be detected by a forehead-attached system where the emitter and detector are on the same side of the subject. In some embodiments, the received light may have been primarily transmitted through the subject. For example, transmitted light may be detected in a fingertip-attached or earlobe-attached sensor.

In some embodiments, the detected light signal received at step 502 may include an ambient light signal component and a signal component corresponding to a wavelength of light emitted by the physiological sensor. In some embodiments, the signal component may correspond to one or more wavelengths of light emitted by the physiological sensor. The ambient signal may be determined, for example, during the period of a light drive cycle when the emitters are not emitting light. For example, the ambient signal may correspond to “off” period 220 of FIG. 2A and the component corresponding to the signal component may correspond to the signal received during a drive pulse, such as drive pulse 202 of FIG. 2A.

In some embodiments, the ambient signal may, for example, include ambient signal 222 of FIG. 2. In some embodiments, the system may subtract ambient signal 222 or a signal derived from ambient signal 222 from the received signal to generate an adjusted signal. The adjusted signal may be used to determine physiological parameters. In some embodiments, the system may determine an ambient signal for sensor-off analysis before generating the adjusted signal. Separation of the ambient signal from the received signal may include, for example, ambient subtractor 162 of FIG. 1. Signal processing of the ambient component and emitted light component may include any suitable components of physiological monitoring system 100 of FIG. 1, physiological monitoring system 310 of FIG. 3, any other suitable components, or any combination thereof.

In some embodiments, the system may use the physiological sensor to emit a photonic signal. The system may emit a photonic signal including one wavelength of light, multiple wavelengths of light, a broad-band spectrum light (e.g., white light), or any combination thereof. For example, the photonic signal may include light from a red LED and light from an IR LED. The emitted photonic signal may be emitted, for example, by light source 130 of FIG. 1, according to a drive signal from light drive circuitry 120. In some embodiments, the emitted photonic signal may include a light drive modulation (e.g., a time division multiplexing, a frequency division multiplexing, or other multiplexing). For example, where the photonic signal includes a red light source and an IR light source, the light drive modulation may include a red drive pulse followed by an “off” period followed by an IR drive pulse followed by an off period. In a further example, where the photonic signal includes an IR light source, the light drive modulation may include a cycling of an IR drive pulse followed by an off period. It will be understood that these drive cycle modulations are merely exemplary and that any suitable drive cycle modulation or combination of modulations may be used. In some embodiments, the photonic signal may include a cardiac cycle modulation, where the brightness, duty cycle, or other parameters of one or more emitters are varied at a rate substantially related to the cardiac cycle.

In some embodiments, the system may adjust or compensate a signal depending in part on the LED drive signal, the detector gain, other suitable system parameters, or any combination thereof. For example, increasing the gain on a detected signal may result in an increased ambient signal. The system may compensate for this increased ambient that is not correlated with a change in the sensor positioning. In a further example, the system may change the LED emitter brightness, resulting in a change in the detected signals. The system may compensate for these changes in the detected signal amplitude to distinguish them from a change in the sensor positioning. It will be understood that the system may make any adjustments in gain, amplification, frequency, wavelength, amplitude, any other suitable adjustments, or any combination thereof. It will be understood that the adjustments may be made to the emitted photonic signal, the received signal, a signal following a number of processing steps, any other suitable signals, or any combination thereof.

Step 504 may include the system processing the detected light signal of step 502 to obtain a first signal (i.e., an AM signal) corresponding to the ambient signal component. In some embodiments, the system may demultiplex the detected light signal to obtain the first signal (e.g., using demultiplexer 154 of system 100. For example, light drive circuitry 120 may be configured to provide a time division multiplexed (TDM) scheduled photonic signal having periods during which an emitter is activated and periods during which no emitter is activated. Demultiplexer 154 may demultiplex the detected light signal based on the TDM schedule. In some embodiments, the first signal may correspond to a first periodic time interval during which no light is emitted.

Step 506 may include the system processing the detected light signal of step 502 to obtain a second signal corresponding to the ambient signal component and the signal component. In some embodiments, the system may demultiplex the detected light signal to obtain the first signal (e.g., using demultiplexer 154 of system 100. For example, light drive circuitry 120 may be configured to provide a time division multiplexed (TDM) scheduled photonic signal having periods during which an emitter is activated and periods during which no emitter is activated. Demultiplexer 154 may demultiplex the detected light signal based on the TDM schedule. In some embodiments, the second signal may correspond to a second periodic time interval during which at least one wavelength of light is emitted (e.g., by light source 130 of system 100).

In step 508, the system may determine at least one reference characteristic based on the first signal (i.e., corresponding to the ambient signal component). The reference characteristic may include a value of the first signal, a level of the first signal, an amplitude of the first signal, a moving average of the first signal, any other suitable characteristic, or any combination thereof. A reference characteristic may be relative, absolute, or any combination thereof. For example, the first signal level may be the absolute amplitude. In a further example, the first signal level may be relative to a baseline value or relative to another signal. Determining the signal level may be performed by any suitable processing equipment described above. The system may apply filtering, smoothing, averaging, any other suitable technique, or any combination thereof to the light signal. For example, the first signal may be filtered to remove noise (e.g., low-pass filtered to substantially reduce high frequency noise). In another example, the first signal may be smoothed or averaged to remove transient signals (e.g., fluctuations over relatively short time scales).

In some embodiments, a reference characteristic may include an initial value of the first signal such as, for example, a value at system startup. In some embodiments, a reference characteristic may include a value derived from a statistical calculation of the first signal. For example, an average value of the first signal may be used as a reference characteristic. In some embodiments, a value of the first signal at a particular time may be used as a reference characteristic. In some embodiments, a reference characteristic may be derived from a value of the first signal. For example, a baseline first signal value may be determined (e.g., such as an initial first signal value), and a threshold may be determined based on the baseline first signal value. In an illustrative example, a reference characteristic may include a threshold T determined using Eq. 1:

T=S_{1,baseline}+ΔT (1)

in which S_{1,baseline} is the baseline value of the first signal, and ΔT is fixed or variable increment used to set the threshold value. In some embodiments, a reference characteristic may include a sequence of values such as, for example, a function, a set of first signal values, or a set of threshold values. Thresholds may be predetermined, set by the user, determined based on historical information, determined based on characteristics related to the patient, determined based on characteristics of the sensor and system, determined based on any other suitable criteria, or any combination thereof. Thresholds may be constant or vary in time. The threshold may include multiple threshold values corresponding to multiple characteristics.

In step 510, the system may compare the second signal to the at least one reference characteristic of step 508. In some embodiments, the system may use one or more threshold values related to the first signal determined at step 508. In some embodiments, step 510 may include determining a difference between a value of the second signal and a reference characteristic. For example, a value of the second signal reaching or crossing a threshold may result in an alarm being triggered, a flag being set, an indication being generated, a signal being generated, any other suitable output, or any combination thereof.

It will be understood that the comparing of step 510 is distinct from determining an attenuation of the signal. For example, determining a change in attenuation of a signal includes comparing the signal at two points in time. In step 510, the second signal may be compared to a threshold at each point in time of the monitoring to determine proximity to the reference characteristic. In an example, an IR signal may be compared to a threshold level corresponding to an ambient baseline.

In step 512, the system may determine whether the physiological sensor is positioned properly. The system may determine that the sensor is not properly positioned based on the comparison of the second signal to the reference characteristic. For example, if the comparison of step 508 indicates that a threshold has been reached or exceeded, then the system may determine that the sensor is not properly positioned.

In some embodiments, a threshold may be set at step 508 during a reset period. For example, the reset period may be triggered by a user to indicate a normal operating state of the system. The normal operating state may include proper positioning of the sensor. The reset mode may include setting a normal level or trend for the first signal and determining a threshold based on that level or trend. In some embodiments, a reset period may be triggered automatically based on time, sensor connections, signal conditions, a physiological condition or event, any other suitable triggers, or any combination thereof.

In some embodiments, where the reference characteristic is a threshold based on first signal level (e.g., determined using Eq. 1), the threshold may be a lower limit on the level of the second signal before a Sensor-Off flag is triggered. The threshold may be a constant level, a moving average, a predetermined pattern, a patterned determined based on user input, a pattern based on historical information, any other suitable threshold, or any combination thereof.

In some embodiments, the comparison of step 510 may include a comparison between multiple signal components derived from a light signal. For example, the first signal level may be compared to the second signal. Comparison may include a subtraction, division, multiplication, integration, any other suitable function, or any combination thereof. Comparisons may also include time-domain comparisons. For example, a level of the first signal may be compared to a moving average of the second signal. In some embodiments, comparing multiple signal components may help identify a Sensor Off or other undesirable system condition from an external, unrelated change. In some embodiments, one or more values of the second signal may be compared to one or more corresponding values of a first signal baseline. For example, the system may determine a maximum difference and a minimum difference between the second signal and the first signal baseline. The system may then compare the maximum difference to the minimum difference to determine whether the sensor is properly positioned (e.g., if the difference is above a predetermined value, then fluctuations are indicated and a Sensor Off condition may be identified).

In some embodiments, the system may use multiple criteria to determine a sensor-off condition. The multiple criteria may be combined using any suitable logic method, algorithmic method, polling method, weighted method, any other suitable methods, or any combination thereof. In some embodiments, the system may determine a confidence value related to the possibility of a sensor-off condition based on the criteria. The presence of a combination of one or more the above conditions (e.g., reaching or crossing a threshold, exhibiting fluctuations) may indicate a Sensor Off condition, or may form part of a Sensor Off algorithm which may also contain other indicators of the Sensor Off condition not presented here.

Metrics based on the above may be used within a polled, logical or weighted method to determine Sensor Off. Those skilled in the art will recognize that the above may be performed using the Red light signal instead of the IR signal. Those skilled in the art will recognize that the above may be performed using the either or both the Red light and IR signals. Those skilled in the art will recognize that a combination of one or more of the above conditions may indicate a Sensor Off condition for any suitable pulse oximeter sensors, including finger sensors, ear sensors, toe sensors. Those skilled in the art will recognize that the above condition may indicate a Sensor Off condition for other sensors which determine regional saturation (rSO₂). Those skilled in the art will recognize that a combination of one or more of the above conditions may indicate a Sensor Off condition for sensors used for purposes other than the determination of SP0₂ or pulse rate. These other purposes may include the determination of respiration rate, respiration effort, continuous non-invasive blood pressure, saturation pattern detection, fluid responsiveness, cardiac output, or other clinical parameter that may be determined using a pulse oximeter sensor system. Those skilled in the art will recognize that the above may be applied to other sensors that depend on the analysis of light levels where an ambient or dark signal may be acquired.

It will be understood that the above described sensor-off detection techniques are merely exemplary and that any suitable signal characteristics or combination of signal characteristics may be used with any suitable thresholds or combination of thresholds to determine a sensor-off condition.

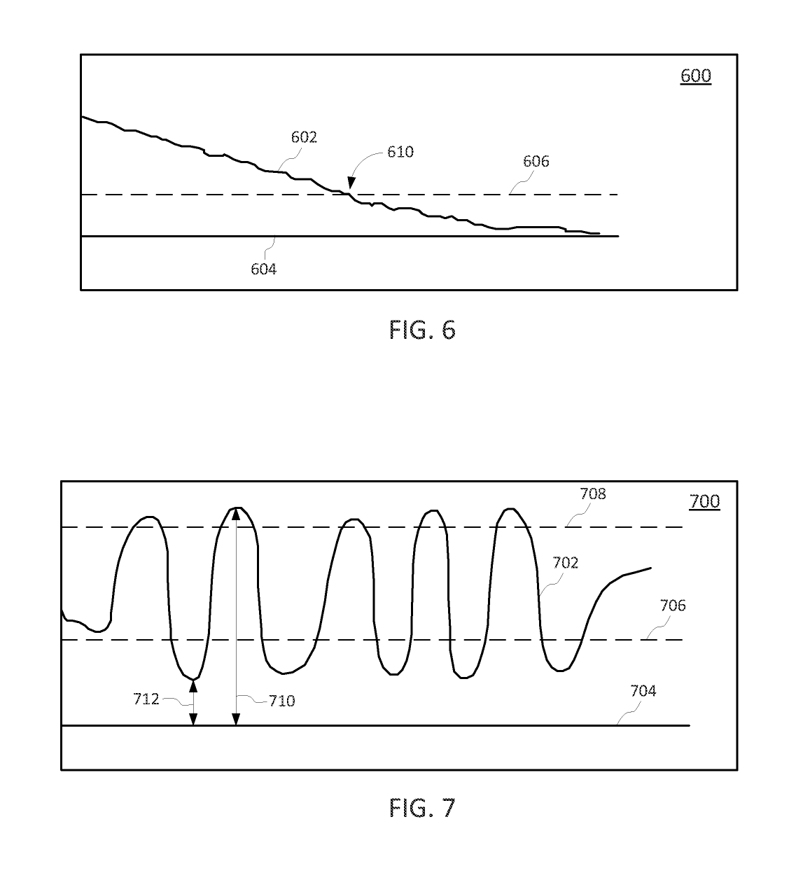

FIGS. 6-8 provide illustrative graphical examples of the techniques of flow diagram 500 of FIG. 5. It will be understood that the disclosed technique may be used without graphing or plotting data, and the plots of FIGS. 6-8 are provided for illustration. FIGS. 6-8 will be discussed in the context of an illustrative IR signal (including the ambient signal) and an illustrative ambient signal, although any suitable signal components may be used in accordance with the present disclosure.

FIG. 6 shows an illustrative plot 600 of an IR signal 602, an ambient baseline signal 604 and a threshold 606, in accordance with some embodiments of the present disclosure. IR signal 602 may correspond to IR-wavelength light provided by an IR LED, for example. IR signal 602 becomes proximal to the ambient baseline signal 604, which is shown illustratively as a constant value, as time progress (i.e., moving from left to right along the abscissa). In the illustrated embodiment, this proximity is indicated by the IR signal value crossing threshold 606 at point 610. The system may identify a Sensor-Off condition based on the threshold crossing at point 610. Threshold 606 may be set based on ambient baseline signal 604 (e.g., using Eq. 1 and the AM signal value when the sensor was first attached or a lower bound AM signal value taken over a recent period of time). Although shown in FIG. 6 as a constant value, ambient baseline signal 604 may be updated from time to time during the monitoring process.

FIG. 7 shows an illustrative plot 700 of a fluctuating IR signal 702, an ambient baseline 704 and thresholds 706 and 708, in accordance with some embodiments of the present disclosure. The intermittent presence of the IR signal below threshold 706, or above threshold 708, or both, may indicate a sensor off condition. An upper bound threshold, a lower bound threshold, or both, may be used to determine whether a sensor is positioned properly. In some embodiments, maximum difference 710 and minimum difference 712 of the IR signal relative to the ambient baseline may also indicate a Sensor Off condition. For example, the system may determine the difference between maximum difference 710 and minimum difference 712, and compare the difference to a threshold. If the difference exceeds the threshold, indicating fluctuations of IR the system may determine that a sensor is not positioned properly.

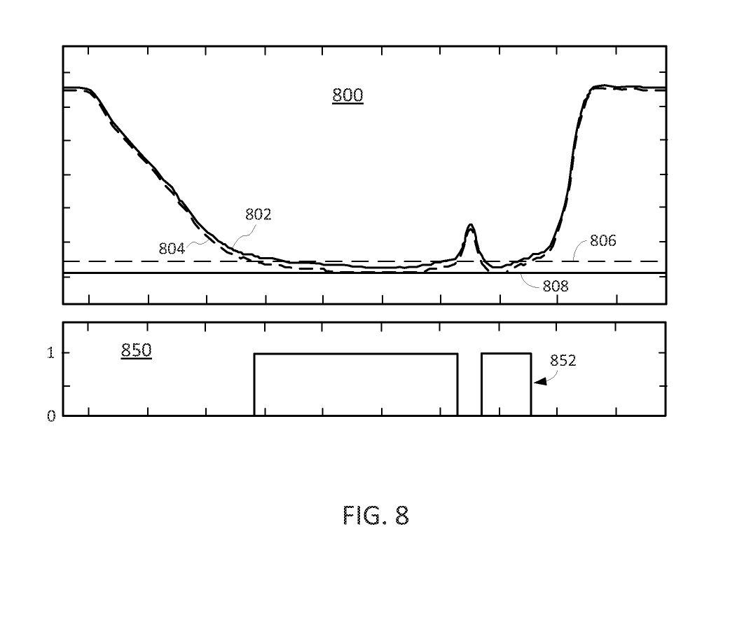

FIG. 8 shows illustrative plots of an IR signal, an ambient signal component, and a threshold, along with a sensor-off flag, in accordance with some embodiments of the present disclosure. Plot 800 shows IR signal 802 and AM signal 804 during a sensor off event in which IR signal 802 has reduced to a value near baseline value 808. Threshold 806, determined based on baseline value 808 (e.g., using Eq. 1), is used by the system to set a flag to unity, indicating a sensor-off event, as shown by flag 852 of plot 850 assuming a value of one. For example, AM signal 804 may be compared to threshold 808 and flag 852 set to a value of one when the signal is below the threshold. Threshold 806 is also used by the system to reset the flag to zero, indicating the end of a Sensor Off event, as shown by flag 852 of plot 850 assuming a value of zero. In some embodiments, plot 850 is plotted on the same x-axis time scale as plot 800.

In some embodiments, the signals of plot 800 show signals in a sensor-off condition for the entire plot. While flag 852 assumes a value of zero for a portion of plot 850, the probe may be in a sensor-off condition for the duration of the plot. It will be understood that the flag assuming a value of one may be indicative of a particular sensor-off identification that may in some embodiments be combined with other techniques of identifying sensor-off conditions. In some embodiments, both IR signal 802 and AM signal 804 are initially relatively high as compared to baseline value 808 because, for example, the detector is detached from a patient and facing a light source. When facing a light source, a large and similar amount of light may reach the detector during all cycles of the light drive signal, for example the cycles illustrated in FIG. 2A. In some embodiments, the level of both IR signal 802 and AM signal 804 may decrease due to partial shielding of the detector because of, for example, covering by bedsheets or clothing. Separation between the IR signal 802 and AM signal 804 may occur due to partial reflection of emitted light reaching the detector when the sensor-off sensor is near, but not attached to, a reflective surface. In some embodiments, changes in the separation may correspond to changes in the amount of reflected light.

In a further example, the duration, magnitude, or occurrence of a threshold crossing may indicate a false-positive (e.g., a sensor is erroneously determined to be improperly positioned). In a further example, a number of threshold crossings may be indicative of a false-positive. In some embodiments, the system may enter a reset period and/or adjust a threshold following a false-positive. In some embodiments, the system may generate an indication (e.g., visual or audial) that a false-positive has occurred. In some embodiments, a system tolerance for false positives may be user selectable or otherwise adjustable depending on, for example, the condition of the patient. For example, a system may be configured so that any threshold crossing triggers a flag signal. In a further example, a system may be configured so that a threshold must be crossed for a certain amount of time or by a certain amount to trigger a flag signal.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for determining whether a physiological sensor is properly positioned on a subject, the method comprising:
receiving a detected light signal, wherein the light signal comprises an ambient light signal component and a signal component corresponding to a wavelength of light emitted by the physiological sensor;
processing the detected light signal, using processing equipment, to generate a first signal corresponding to the ambient light signal component;
processing the detected light signal, using the processing equipment, to generate a second signal corresponding to the ambient light signal component and the signal component;
determining, using the processing equipment, at least one reference characteristic based on the first signal;
comparing, using the processing equipment, the second signal to the at least one reference characteristic; and
determining, using the processing equipment, whether the physiological sensor is properly positioned based on the comparison.

2. The method of claim 1, wherein the physiological sensor comprises a pulse oximetry sensor.

3. The method of claim 1, wherein determining the at least one reference characteristic comprises generating a threshold based on at least one particular value of the first signal.

4. The method of claim 3, wherein the at least one particular value of the first signal comprises an initial value of the first signal.

5. The method of claim 3, wherein the at least one particular value of the first signal comprises a value selected from the first signal at a particular time interval.

6. The method of claim 3, wherein determining whether the physiological sensor is properly positioned comprises determining whether the second signal crosses the threshold.

7. The method of claim 1, wherein the at least one reference characteristic comprises a first reference characteristic corresponding to a first value of the first signal and a second reference characteristic corresponding to a second value of the first signal, the method further comprising:
determining a first difference between the second signal and the first value;
determining a second difference between the second signal and the second value; and
determining a difference between the first difference and the second difference, wherein determining whether the physiological sensor is properly positioned is based on the determined difference.

8. The method of claim 7, wherein the first difference corresponds to a maximum difference between the second signal and the first signal, and the second difference corresponds to a minimum difference between the second signal and the first signal.

9. The method of claim 1, further comprising setting a sensor-off flag when it is determined that the physiological sensor is not properly positioned.

10. The method of claim 9, further comprising resetting the sensor-off flag when it is subsequently determined that the physiological sensor is properly positioned.

11. A system for determining whether a physiological sensor is properly positioned on a subject, the system comprising:
processing equipment configured to:
receive a detected light signal, wherein the light signal comprises an ambient light signal component and a signal component corresponding to a wavelength of light emitted by the physiological sensor;
process the detected light signal to generate a first signal corresponding to the ambient light signal component;
process the detected light signal to generate a second signal corresponding to the ambient light signal component and the signal component;
determine at least one reference characteristic based on the first signal;
compare the second signal to the at least one reference characteristic; and
determine whether the physiological sensor is properly positioned based on the comparison.

12. The system of claim 11, wherein the physiological sensor comprises a pulse oximetry sensor.

13. The system of claim 11, wherein the processing equipment is further configured to determine the at least one reference characteristic by generating a threshold based on at least one particular value of the first signal.

14. The system of claim 13, wherein the at least one particular value of the first signal comprises an initial value of the first signal.

15. The system of claim 13, wherein the at least one particular value of the first signal comprises a value selected from the first signal at a particular time interval.

16. The system of claim 13, wherein the processing equipment is further configured to determine whether the physiological sensor is properly positioned by determining whether the second signal crosses the threshold.

17. The system of claim 11, wherein the at least one reference characteristic comprises a first reference characteristic corresponding to a first value of the first signal and a second reference characteristic corresponding to a second value of the first signal, wherein the processing equipment is further configured to:
determine a first difference between the second signal and the first value;
determine a second difference between the second signal and the second value;
determine a difference between the first difference and the second difference; and
determine whether the physiological sensor is properly positioned further based on the determined difference.

18. The system of claim 17, wherein the first difference corresponds to a maximum difference between the second signal and the first signal, and the second difference corresponds to a minimum difference between the second signal and the first signal.

19. The system of claim 11, wherein the processing equipment is further configured to set a sensor-off flag when it is determined that the physiological sensor is not properly positioned.

20. The system of claim 19, wherein the processing equipment is further configured to reset the sensor-off flag when it is subsequently determined that the physiological sensor is properly positioned.
